(19) United States
(12) Patent Application Publication
(10) Pub. No.: US 2013/0289049 A1
(43) Pub. Date: Oct. 31, 2013

(54) ACID ADDITION SALTS OF THE 2-[2-[[(4-METHOXY-2,6-DIMETHYLPHENYL)SULFONYL]-(METHYL)AMINO]ETHOXY]-N-METHYL-N-[3-(4-METHYLPIPERAZIN-1-YL)CYCLOHEXYL] ACETAMIDE AND THE USE THEREOF AS BRADYKININ B1 RECEPTORANTAGONISTS

(71) Applicants: Thorsten Pachur, Biberach an der Riss (DE); Waldemar Pfrengle, Biberach an der Riss (DE); Manfred Birk, Biberach an der Riss (DE); Juergen Schnaubelt, Warthausen (DE); Ulrike Werthmann, Biberach an der Riss (DE)

(72) Inventors: Thorsten Pachur, Biberach an der Riss (DE); Waldemar Pfrengle, Biberach an der Riss (DE); Manfred Birk, Biberach an der Riss (DE); Juergen Schnaubelt, Warthausen (DE); Ulrike Werthmann, Biberach an der Riss (DE)

(73) Assignee: Boehringer Ingelheim International GmbH, Ingelheim am Rhein (DE)

(21) Appl. No.: 13/814,006

(22) PCT Filed: Aug. 4, 2011
(86) PCT No.: PCT/EP2011/063413

§ 371 Date: Jul. 10, 2013

(30) Foreign Application Priority Data
Aug. 5, 2010 (EP) 10172018.3

(51) Int. Cl. C07D 295/155

(52) U.S. Cl. CPC C07D 295/155
USPC 514/255.03; 544/400

(57) ABSTRACT

The present invention relates to the new acid addition salts AB of the following free base of formula A

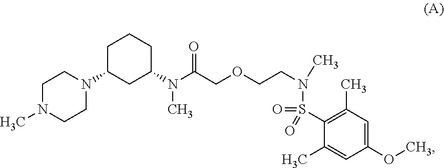 [SMILES: COc1cc(C)c(S(=O)(=O)N(C)CCOCC(=O)N(C)[C@H]2CCC[C@@H](N3CCN(C)CC3)C2)c(C)c1]

or the enantiomer thereof with a physiologically acceptable acid B which is selected from among hydrochloric acid, fumaric acid and tartaric acid, as well as the polymorphs, hydrates and solvates thereof.

The present invention relates to the new acid addition salts AB of the following free base of formula A

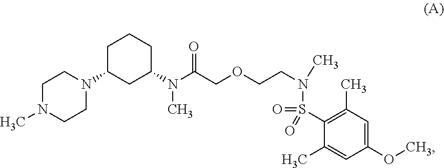 [SMILES: COc1cc(C)c(S(=O)(=O)N(C)CCOCC(=O)N(C)[C@H]2CCC[C@@H](N3CCN(C)CC3)C2)c(C)c1]

or the enantiomer thereof with a physiologically acceptable acid B, which is selected from among hydrochloric acid, fumaric acid and tartaric acid, as well as the polymorphs, hydrates and solvates thereof.

Preferably the acid addition salts according to the invention are present in crystalline form.

### BACKGROUND TO THE INVENTION

1. Technical Field

The present invention relates to B1-antagonists, which are present in the form of stable crystalline derivatives and are suitable for the treatment or prevention of acute pain, visceral pain, neuropathic pain, inflammatory and pain receptor-mediated pain, tumour pain as well as headaches.

2. Prior Art

The compound of formula A, the enantiomer thereof and the preparation thereof have already been described in European Patent Application EP 2 025 673.

### DETAILED DESCRIPTION OF THE INVENTION

The pharmacologically valuable properties of the compounds according to the invention constitute the basic prerequisite for effective use of the compound as a medicament. However, an active substance also has to meet further requirements, in order to be allowed to be used as a medicament. These parameters are to a large extent connected to the physicochemical nature of the active substance.

Without being restricted thereto, examples of these parameters are the stability of action of the starting material under different ambient conditions, the stability during manufacture of the pharmaceutical formulation and the stability in the final compositions of the medicament. The active substance used to produce the medicament compositions should therefore have a high stability, which must also be guaranteed even under different, fluctuating ambient conditions. This is absolutely essential to prevent the use of medicament compositions in which for example breakdown products of the active substance are present in addition to the active substance itself. In such a case a content of active substance appearing in pharmaceutical formulations might be lower than specified.

The absorption of moisture reduces the content of pharmaceutically active substance because of the weight increase caused by the uptake of water. Pharmaceutical compositions with a tendency to absorb moisture have to be protected from moisture during storage, for example by the addition of suitable drying agents or by storing the pharmaceutical composition in an environment that is protected from moisture. In addition the uptake of moisture may reduce the content of active substance during production if the medicament is exposed to the environment without any protection from moisture. Therefore a pharmaceutically active substance should preferably be only slightly hygroscopic.

As the same crystalline modification of the active substance always has to be guaranteed for the pharmaceutical quality of a medicament formulation, increased demands are to be made on the stability and properties of the crystalline active substance against this background. It is particularly desirable to provide the active substance in the form of a unified and clearly defined crystal modification. It is also particularly desirable to provide the active substance in a crystalline form which is characterised by a high degree of stability even when stored for long periods. The less tendency a crystal modification has to absorb moisture, for example, the greater the physical stability of its crystalline structure.

As the crystal modification of an active substance is important to the reproducible active substance content of a preparation, there is a need to clarify as far as possible any existing polymorphism of an active substance present in crystalline form. If different polymorphous modifications of an active substance occur, it should be ensured that the crystalline modification of the substance does not change in the medicament preparation obtained subsequently. Otherwise this could have an adverse effect on the reproducible efficacy of the medicament. Against this background, active substances which are characterised only by low polymorphism are preferred.

Another criterion which is of exceptional importance in some cases, depending on the choice of the formulation or the choice of the method of preparation of the formulation, is the solubility of the active substance. If for example medicament solutions are prepared (e.g. for infusions) it is essential that the active substance should be adequately soluble in physiologically acceptable solvents. For medicaments to be administered orally it is also very important that the active substance should be sufficiently soluble.

The problem on which the present invention is based is to provide a pharmaceutically active substance which is not only characterised by a high pharmacological efficacy but also meets the above-mentioned physicochemical requirements as far as possible.

Surprisingly, it has been found that the above-mentioned problem is solved by the enantiomerically pure compounds according to the invention.

The acid addition salts according to the invention are predominantly characterised by their stable crystalline form, whereas the free base is present as an oil.

In a first aspect the present invention relates to the new acid addition salts AB of the following free base of formula A

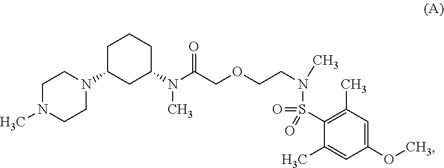 [SMILES: COc1cc(C)c(S(=O)(=O)N(C)CCOCC(=O)N(C)[C@H]2CCC[C@@H](N3CCN(C)CC3)C2)c(C)c1]

or the enantiomer thereof with a physiologically acceptable acid B, which is selected from among hydrochloric acid, fumaric acid and tartaric acid, as well as the polymorphs, hydrates and solvates thereof.

The acid addition salts of the present invention are obtained in enantiomerically pure form.

The term “enantiomerically pure” within the scope of the present invention describes compounds of formula A with a physiologically acceptable acid B, which is present in an enantiomeric purity of at least 85% ee, preferably at least 90% ee, particularly preferably ≧95% ee. The term “ee” (enantiomeric excess) is known in the art and describes the optical purity of chiral compounds.

In a second aspect the present invention relates to the following compounds:
(1) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-dihydrochloride (1),
(2) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2),
(3) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3),
(4) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4),
(5) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-difumarate (5) and
(6) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-difumarate (6),
   the solvates and the hydrates thereof.

The compounds according to the invention are characterised by a high level of stability and dissolve very easily in physiologically acceptable solvents.

In a third aspect the present invention relates to the previously mentioned compounds in crystalline form.

The crystalline salts (3), (4), (5) and (6) are characterised in each case by a characteristic melting point which has been determined by Differential Scanning calorimetry (DSC: evaluated by onset temperature or peak maximum, heating rate: 10° C./min). The values of the individual compounds listed in Table 1 were determined using a DSC 822 made by Mettler Toledo or Q1000 produced by TA Instruments.

TABLE 1
melting points of the crystalline salts according to the invention

| | melting point Tₘₚ. |
|---|---|
| compound | [° C.] |
| (3) | 167 ± 5 (onset) |
| (4) | 167 ± 5 (onset) |
| (5) | 153 ± 5 (onset) |
| (6) | 152 ± 5 (onset) |

In a fourth aspect the present invention relates to crystalline salts according to the invention, in each case characterised by their characteristic melting point.

The melting point is dependent on the degree of purity of a compound and rises as the purity increases. This means that the compounds of the present invention may have a higher or lower melting point than is specified in each case.

In a fifth aspect the invention relates to the crystalline compound (3), characterised by a melting point of T_{mp.}=167±5° C.

In a sixth aspect the invention relates to the crystalline compound (4), characterised by a melting point of T_{mp.}=167±5° C.

In a seventh aspect the invention relates to the crystalline compound (5), characterised by a melting point of T_{mp.}=153±5° C.

In an eighth aspect the invention relates to the crystalline compound (6), characterised by a melting point of T_{mp.}=152±5° C.

The crystalline forms of the individual salts according to the invention were investigated more closely by X-ray powder diffraction. The diagrams obtained are shown in FIGS. 1 to 4.

Tables 2, 3, 4 and 5 below show the data obtained in the analyses carried out.

TABLE 2
X-ray powder reflections and intensities (normalised) of compound (1).

| d value | 2-theta | relative intensity |
|---|---|---|
| [Å] | [°] | [%] |
| 5.95 | 14.89 | 100 |
| 5.62 | 15.74 | 100 |
| 4.65 | 19.07 | 85 |

TABLE 3
X-ray powder reflections and intensities (normalised) of compound (2).

| d value | 2-theta | relative intensity |
|---|---|---|
| [Å] | [°] | [%] |
| 5.95 | 14.89 | 100 |
| 5.62 | 15.75 | 71 |
| 4.65 | 19.07 | 65 |

TABLE 4
X-ray powder reflections and intensities (normalised) of compound (3).

| d value | 2-theta | relative intensity |
|---|---|---|
| [Å] | [°] | [%] |
| 4.94 | 17.94 | 100 |
| 5.15 | 17.22 | 76 |
| 5.22 | 16.98 | 34 |

TABLE 5
X-ray powder reflections and intensities (normalised) of compound (4).

| d value | 2-theta | relative intensity |
|---|---|---|
| [Å] | [°] | [%] |
| 4.94 | 17.92 | 100 |
| 5.15 | 17.20 | 73 |
| 5.22 | 16.97 | 33 |

In Tables 2 to 5 above the value “2Θ±0.05 [°]” denotes the diffraction angle in degrees and the value “d (hkl) [Å]” for the measured distances in Å between the lattice planes.

The X-ray powder diagrams of compounds (1), (2), (3) and (4) were recorded within the scope of the present invention using a STOE-STADI P diffractometer in transmission mode, fitted with a site-sensitive detector (OED) and a Cu anode as the X-ray source with monochromatic CuK_{α1} radiation (λ=1.54056 Å, 40 kV, 40 mA).

In accordance with the findings shown in Table 2 the present invention relates to crystalline 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-dihydrochloride (1), characterised in that in the X-ray powder diagram it has, inter alia, the characteristic values d=5.95 Å, 5.62 Å and 4.65 Å.

According to the findings shown in Table 3 the present invention relates to crystalline 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2), characterised in that in the X-ray powder diagram it has, inter alia, the characteristic values d=5.95 Å, 5.62 Å and 4.65 Å.

According to the findings shown in Table 4 the present invention relates to crystalline 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3), characterised in that in the X-ray powder diagram it has, inter alia, the characteristic values d=4.94 Å, 5.15 Å and 5.22 Å.

According to the findings shown in Table 5 the present invention relates to crystalline 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4), characterised in that in the X-ray powder diagram it has, inter alia, the characteristic values d=4.94 Å, 5.15 Å and 5.22 Å.

The crystalline form of compound (3) is further characterised by the following lattice parameters:
(i) orthorhombic system;
(ii) spatial group P2₁2₁2₁ with the cell parameters a=6.251(2) Å, b=8.127(3) Å, c=66.52(2) Å and a cell volume of 3380(1) Å³

The values are obtained by labelling an X-ray powder diagram recorded at ambient temperature and using CuK_{α1} radiation.

Table 6 lists the labelled peaks (±0.05° 2Θ) with their relative intensities.

TABLE 6
Labelled XRPD peaks (up to 40° 2Θ) including relative
intensities of compound (3)
(abbreviations used: obs. = observed, rep. = reported)

| 2 Θ | dₕₖₗ | intensity | labelling | | | 2 ⊖_{obs.} − 2 ⊖_{rep.} |
|---|---|---|---|---|---|---|
| [°] | [Å] | I/I_{O} [%] | h | k | l | [°] |
| 5.32 | 16.61 | 4 | 0 | 0 | 4 | 0.007 |
| 10.64 | 8.31 | 12 | 0 | 0 | 8 | 0.007 |
| 10.98 | 8.05 | 7 | 0 | 1 | 1 | 0.017 |
| 11.20 | 7.89 | 17 | 0 | 1 | 2 | 0.004 |
| 12.12 | 7.30 | 20 | 0 | 1 | 4 | 0.007 |
| 12.75 | 6.94 | 1 | 0 | 1 | 5 | <0.001 |
| 13.51 | 6.55 | 18 | 0 | 1 | 6 | 0.01 |
| 14.35 | 6.17 | 6 | 0 | 1 | 7 | 0.024 |
| 14.72 | 6.01 | 13 | 1 | 0 | 3 | 0.01 |
| 15.14 | 5.85 | 13 | 1 | 0 | 4 | 0.01 |
| 15.66 | 5.65 | 26 | 1 | 0 | 5 | 0.009 |
| 15.98 | 5.54 | 31 | 0 | 0 | 12 | 0.005 |
| 16.23 | 5.46 | 11 | 0 | 1 | 9 | 0.03 |
| 16.98 | 5.22 | 34 | 1 | 0 | 7 | 0.013 |
| 17.22 | 5.15 | 76 | 0 | 1 | 10 | 0.003 |
| 17.94 | 4.94 | 100 | 1 | 1 | 0 | 0.053 |
| 18.29 | 4.85 | 22 | 0 | 1 | 11 | 0.015 |
| 18.62 | 4.76 | 19 | 1 | 0 | 9 | 0.047 |
| 19.10 | 4.64 | 6 | 1 | 1 | 5 | 0.005 |
| 19.61 | 4.52 | 29 | 1 | 1 | 6 | 0.002 |
| 20.19 | 4.39 | 5 | 1 | 1 | 7 | −0.001 |
| 20.42 | 4.35 | 3 | 1 | 0 | 11 | −0.001 |
| 20.86 | 4.26 | 12 | 1 | 1 | 8 | 0.005 |
| 21.41 | 4.15 | 29 | 0 | 0 | 16 | 0.056 |
| 21.59 | 4.11 | 13 | 1 | 1 | 9 | 0.012 |
| 21.91 | 4.05 | 11 | 0 | 2 | 0 | 0.055 |
| 22.35 | 3.97 | 7 | 1 | 1 | 10 | −0.006 |
| 22.86 | 3.89 | 1 | 0 | 1 | 15 | 0.037 |
| 23.23 | 3.83 | 4 | 1 | 1 | 11 | 0.038 |
| 23.49 | 3.78 | 3 | 1 | 0 | 14 | −0.006 |
| 23.80 | 3.74 | 2 | 0 | 2 | 7 | 0.002 |
| 24.05 | 3.70 | 9 | 0 | 1 | 16 | 0.03 |
| 24.60 | 3.62 | 9 | 1 | 0 | 15 | 0.009 |
| 25.00 | 3.56 | 11 | 0 | 2 | 9 | 0.011 |
| 25.70 | 3.46 | 5 | 0 | 2 | 10 | 0.027 |
| 25.97 | 3.43 | 3 | 1 | 1 | 14 | 0.013 |
| 26.43 | 3.37 | 23 | 0 | 2 | 11 | 0.029 |
| 26.96 | 3.30 | 8 | 1 | 1 | 15 | 0.001 |
| 27.36 | 3.26 | 1 | 1 | 2 | 6 | −0.005 |
| 27.99 | 3.18 | 4 | 1 | 1 | 16 | 0.001 |
| 28.28 | 3.15 | 5 | 1 | 2 | 8 | −0.004 |
| 29.04 | 3.07 | 15 | 0 | 1 | 20 | 0.057 |
| 29.74 | 3.00 | 2 | 0 | 2 | 15 | −0.056 |
| 30.12 | 2.96 | 1 | 2 | 0 | 7 | 0.051 |
| 30.46 | 2.93 | 4 | 1 | 0 | 20 | 0.037 |
| 30.66 | 2.91 | 8 | 2 | 1 | 0 | 0.035 |
| 31.24 | 2.86 | 5 | 1 | 1 | 19 | −0.013 |
| 31.58 | 2.83 | 6 | 0 | 1 | 22 | 0.038 |
| 32.48 | 2.75 | 1 | 2 | 1 | 8 | −0.023 |
| 33.10 | 2.70 | 3 | 0 | 3 | 1 | 0.036 |
| 33.50 | 2.67 | 6 | 0 | 3 | 4 | 0.008 |
| 34.08 | 2.63 | 3 | 0 | 3 | 6 | 0.037 |
| 34.79 | 2.58 | 2 | 0 | 3 | 8 | −0.01 |
| 35.80 | 2.51 | 2 | 0 | 3 | 10 | 0.045 |
| 36.11 | 2.49 | 4 | 2 | 1 | 14 | 0.012 |
| 36.33 | 2.47 | 4 | 0 | 3 | 11 | 0.017 |
| 36.78 | 2.44 | 10 | 1 | 3 | 5 | 0.023 |
| 37.40 | 2.40 | 4 | 1 | 3 | 7 | 0.03 |
| 37.73 | 2.38 | 3 | 1 | 3 | 8 | −0.01 |
| 38.29 | 2.35 | 4 | 2 | 2 | 9 | 0.001 |
| 38.72 | 2.32 | 4 | 2 | 2 | 10 | −0.033 |
| 39.59 | 2.27 | 4 | 2 | 0 | 20 | 0.058 |

In another aspect the present invention relates to crystalline 2-{2-[[(4-methoxy-2,6-dimethylphenyl)sulphonyl]-(methyl)amino]ethoxy}-N-methyl-N-[(1S,3R)-3-(4-methyl-piperazin-1-yl)cyclohexyl]acetamide-(L)-tartrate (3), characterised in that the crystals are present in an orthorhombic system.

The present invention further relates to pharmaceutical compositions, characterised in that they contain the crystalline, enantiomerically pure compound AB according to the invention. Preferably these compositions are used for the treatment of acute pain, visceral pain, neuropathic pain, inflammatory and pain receptor-mediated pain, tumour pain as well as headaches. The present invention further relates to the use of the crystalline, enantiomerically pure compound AB for preparing a pharmaceutical composition for the treatment of acute pain, visceral pain, neuropathic pain, inflammatory and pain receptor-mediated pain, tumour pain as well as headaches.

The present invention preferably relates to the use of the above-mentioned crystalline and enantiomerically pure compounds of formula AB for preparing a pharmaceutical composition for the treatment of acute pain, visceral pain, neuropathic pain, inflammatory and pain receptor-mediated pain, tumour pain as well as headaches.

### Preparation Methods

The salts of the compound of general formula I are prepared by methods known in principle. The methods listed in the “Handbook of Pharmaceutical Salts” (Eds. P. Heinrich Stahl, Camille G. Wermuth, Wiley-VHC 2002) have proved particularly suitable.

In another aspect the present invention relates to a process for preparing the acid addition salts according to the invention, comprising the following steps:
(a) reaction of 3,5-dimethylanisol of formula II

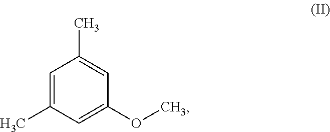

with chlorosulphonic acid;
(b) reaction of 4-methoxy-2,6-dimethylsulphonyl chloride of formula III obtained in step (a)

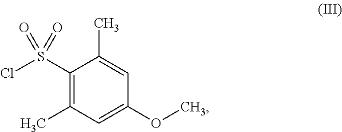

with the compound of formula IV

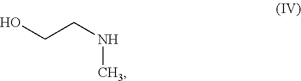

(c) reaction of the compound of formula V obtained in step (b)

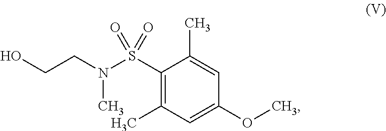 [SMILES: COc1cc(C)c(S(=O)(=O)N(C)CCO)c(C)c1]

with a compound of general formula VI

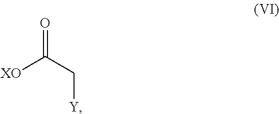

wherein X denotes a hydrogen atom, an alkali metal, for example lithium, sodium or potassium, or a C₁₋₄-alkyl group, but preferably sodium, and Y denotes a halogen atom, for example chlorine or bromine, preferably chlorine;
(d) optionally recrystallisation of a compound of general formula VII obtained in step (c)

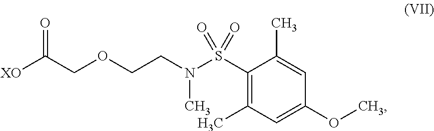

wherein X denotes a hydrogen atom, an alkali metal, for example lithium, sodium or potassium, or a C₁₋₄-alkyl group, but preferably sodium, from a solvent;
(e) coupling a compound of general formula VIII obtained in step (c) or (d)

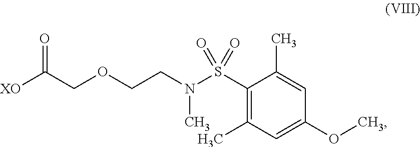

wherein X denotes a hydrogen atom, an alkali metal, for example lithium, sodium or potassium, or a C₁₋₄-alkyl group, but preferably sodium, with a compound of formula IX

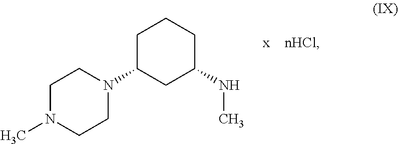

or the enantiomer thereof, wherein n denotes one of the numbers 0, 1, 2 or 3;
(f) optionally isolating a compound of formula A obtained in step (d)

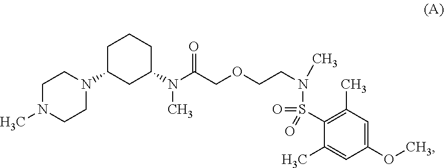 [SMILES: COc1cc(C)c(S(=O)(=O)N(C)CCOCC(=O)N(C)[C@H]2CCC[C@@H](N3CCN(C)CC3)C2)c(C)c1]

or the enantiomer thereof;
(g) dissolving the compound of formula A obtained in step (e) or (f) or the enantiomer thereof in a polar solvent;
(h) adding a physiologically acceptable acid B which is selected from among hydrochloric acid, fumaric acid and tartaric acid, optionally dissolved in a polar solvent; and
(i) crystallising out an acid addition salt AB, optionally by the addition of an antisolvent, by cooling, distilling or seeding.

In the reaction in step (a) preferably 1.0 equivalents 3,5-dimethylanisol of formula II are reacted with 1.5 to 2.5 equivalents, preferably 1.8 to 2.2 equivalents, of chlorosulphonic acid.

The reaction may take place in a solvent which is selected from among dichloromethane, chloroform, carbon tetrachloride and 1,2-dichloroethane. The solvent may be used in an amount of from 0.25 to 1.25 L/mol, preferably 0.60 to 0.90 L/mol of 3,5-dimethylanisol used.

Preferably the reaction is carried out at low temperature, for example between −45° C. and 0° C., preferably between −40° C. and 0° C., more preferably between −35° C. and −10° C.

In the reaction in step (b) preferably 1.0 equivalents 2,6-dimethyl-4-methoxy-sulphonyl chloride of formula III are reacted with 1.5 to 2.5 equivalents, preferably 1.8 to 2.2 equivalents, of a compound of general formula IV.

The reaction may take place in a solvent selected from among dichloromethane, chloroform, carbon tetrachloride and 1,2-dichloroethane. The solvent may be used in an amount of from 0.25 to 1.25 L/mol, preferably 0.5 to 1.0 L/mol of the 2,6-dimethyl-4-methoxy-sulphonyl chloride used.

Preferably the reaction is carried out at a temperature that is below ambient temperature, for example between −0° C. and 20° C., preferably between 5° C. and 15° C.

In the reaction in step (c) preferably 1.0 equivalents of a compound of general formula V are reacted with 1.1 to 2.5 equivalents, preferably 1.4 to 1.7 equivalents, of a compound of general formula VI.

The reaction may be carried out in a solvent selected from among acetonitrile, tetrahydrofuran, methyltetrahydrofuran, acetone, toluene, xylene, dichloromethane and chloroform. The solvent may be used in an amount of from 0.5 to 3 L/mol, preferably 1.2 to 1.7 L/mol of the compound of general formula V used.

A base may also be added to the reaction mixture. The base may be selected from among potassium tert. butoxide, potassium carbonate, sodium carbonate, lithium carbonate, sodium hydride, sodium methoxide and sodium ethoxide, preferably potassium tert. butoxide. It may be added in an amount of from 1.2 to 2.0 equivalents, preferably 1.3 to 1.6 equivalents, based on the amount of compound of general formula V used.

The compound of general formula VII obtained in step (c) may be purified, before the reaction described in step (e), by recrystallisation from a solvent selected from among water, tetrahydrofuran, methyltetrahydrofuran, acetone or the mixtures thereof.

In the coupling in step (e) preferably 1.0 equivalents of a compound of general formula VIII are reacted with 1.0 to 1.5 equivalents, preferably 1.0 to 1.2 equivalents, of a compound of general formula IX or the enantiomer thereof.

The reaction may be carried out in a solvent that is selected from among tetrahydrofuran, methyltetrahydrofuran, dichloromethane, chloroform, toluene, ethyl acetate, isopropylacetate and dioxane. The solvent may be used in an amount of from 1.2 to 2 L/mol, preferably 1.4 to 1.8 L/mol of the compound of general formula VIII used.

A base may also be added to the reaction mixture. The base may be selected from among potassium tert. butoxide, sodium hydroxide, potassium hydroxide, potassium carbonate, sodium carbonate, triethylamine, diisopropylethylamine and diazabicyclo[5.4.0]undec-7-ene (DBU), preferably potassium tert. butoxide. It may be added in an amount of from 3 to 4 equivalents, preferably 3.3 to 3.8 equivalents, based on the amount of compound of general formula VIII used.

Moreover, a coupling reagent may be added to the reaction mixture. The coupling reagent may be selected from among propanephosphonic anhydride, thionyl chloride, N,N,N′,N′-tetramethyl-O-(benzotriazol-1-yl)-uronium tetrafluoroborate, carbodiimide and 1,1′-carbonyldiimidazole; propanephosphonic anhydride is preferably used according to the invention.

Preferably the reaction is carried out at elevated temperature, for example between 40° C. and 60° C.

The compound of general formula IX or the enantiomer thereof may be prepared by a method as described in WO 2010/017850.

The isolation of a compound of formula A or the enantiomer thereof as described in (f) is preferably carried out by evaporation to dryness or crystallisation from water or dichloromethane, methanol, ethanol, propanol, butanol, isopropyl acetate, ethyl acetate, tetrahydrofuran, methyltetrahydrofuran, dioxane, methylisobutylketone, toluene, xylene or mixtures of these solvents, while water, ethanol, tetrahydrofuran, ethyl acetate, methylisobutylketone and toluene or the mixtures thereof are preferably used.

The dissolving of a compound of formula A or the enantiomer thereof obtained in step (e) or (f) takes place in a polar or non-polar solvent, preferably in water or methanol, ethanol, isopropanol, 1-butanol, acetonitrile, acetone, tetrahydrofuran, ethyl acetate, methylisobutylketone, toluene or mixtures of these solvents.

A physiologically acceptable acid B, selected from among hydrogen chloride, fumaric acid and D- or L-tartaric acid, may be added, as described in step (h), either in substance or dissolved in a solvent. The acid B may be used in an amount of from 1.0 to 2.4 equivalents, preferably 2.0 to 2.2 equivalents when a salt is formed with hydrogen chloride or fumaric acid and preferably 1.0 to 1.2 equivalents when a salt is formed with D- or L-tartaric acid, based on the amount of compound of formula A used or the enantiomer thereof. The solvent may be selected from among water, methanol, ethanol, isopropanol, 1-butanol, acetonitrile, acetone, tetrahydrofuran, ethyl acetate, methylisobutylketone or toluene as well as the mixtures thereof. It may be used in an amount of from 0.01 to 10.0 L/mol, preferably 0.02 to 5.0 L/mol of the compound of formula A used or the enantiomer thereof.

The crystallising out described in step (i) is carried out according to the invention with a controlled, stepwise lowering of the temperature and may take place with or without seeding.

The preparation method described may also be used on an industrial scale for preparing large quantities of substance.

### Terms and Definitions Used

By the term “C₁₋₄-alkyl” (including those that are part of other groups) are meant alkyl groups with 1, 2, 3 or 4 carbon atoms. Examples include: methyl, ethyl, n-propyl, iso-propyl, n-butyl, iso-butyl, sec-butyl and tert-butyl. Unless stated otherwise, the definitions propyl and butyl include all the possible isomeric forms of the groups in question. Thus, for example, propyl includes n-propyl and iso-propyl, butyl includes iso-butyl, sec-butyl and tert-butyl.

In another aspect the present invention relates to the use of the new salts as medicaments, on account of their pharmaceutical efficacy.

Description of the Method of Binding the cynoBK1-Receptor

CHO cells that express the cynomolgus BK1-receptor are cultivated in “HAM'S F-12 Medium”. The medium is removed from confluent cultures, the cells are washed with PBS buffer, scraped off or detached using Versene and isolated by centrifuging. Then the cells are homogenised in suspension, the homogenate is centrifuged and resuspended. After the protein content has been determined 200 μl of the homogenate (50 to 250 μg protein/assay) are incubated for 60-180 minutes at ambient temperature with 0.5 to 5.0 nM kallidin (DesArg10,Leu9), [3,4-Prolyl-3,43H(N)] and increasing concentrations of the test substance in a total volume of 250 μl. The incubation is stopped by rapid filtration through GF/B glass fibre filters that have been pre-treated with polyethyleneimine (0.3%). The radioactivity bound to the protein is measured with a TopCount NXT. The radioactivity bound in the presence of 1.0 μM kallidin (DesArg10) is defined as non-specific binding. The concentration binding curve may be analysed using computer-aided non-linear curve fitting to determine the corresponding Kᵢ values for the test substance.

### Indications

In view of their pharmacological properties, the acid addition salts according to the invention are suitable for treating diseases and symptoms of diseases caused at least to some extent by stimulation of bradykinin-B1 receptors, or in which antagonisation of the bradykinin-B1 receptor can bring about an improvement in symptoms.

In a further aspect the present invention encompasses the acid addition salts AB according to the invention for use as medicaments.

In view of their pharmacological effect the substances are suitable for the treatment of
acute pain such as for example toothache, peri- and postoperative pain, traumatic pain, muscle pain, the pain caused by burns, sunburn, trigeminal neuralgia, pain caused by colic, as well as spasms of the gastro-intestinal tract or uterus;
visceral pain such as for example chronic pelvic pain, gynaecological pain, pain before and during menstruation, pain caused by pancreatitis, peptic ulcers, interstitial cystitis, renal colic, cholecystitis, prostatitis, angina pectoris, pain caused by irritable bowel, non-ulcerative dyspepsia and gastritis, prostatitis, non-cardiac thoracic pain and pain caused by myocardial ischaemia and cardiac infarct;
neuropathic pain such as for example painful neuropathies, pain of diabetic neuropathy, AIDS-associated neuropathic pain, non-herpes-associated neuralgia, postherpic neuralgia, nerve damage, cerebro-cranial trauma, pain of nerve damage caused by toxins or chemotherapy, phantom pain, pain of multiple sclerosis, nerve root tears and painful traumatically-caused damage to individual nerves, as well as central pain such as for example pain after stroke, spinal injuries or tumours;
inflammatory/pain receptor-mediated pain in connection with diseases such as for example osteoarthritis, rheumatoid arthritis, rheumatic fever, tendo-synovitis, bursitis, tendonitis, gout and gout-arthritis, traumatic arthritis, vulvodynia, damage to and diseases of the muscles and fascia, juvenile arthritis, spondylitis, psoriasis-arthritis, myositides, dental disease, influenza and other viral infections such as colds, systemic lupus erythematodes or pain caused by burns;
tumour pain associated with cancers such as for example lymphatic or myeloid leukaemia, Hodgkin's disease, non-Hodgkin's lymphomas, lymphogranulomatosis, lymphosarcomas, solid malignant tumours and extensive metastases;
headache diseases of various origins, such as for example cluster headaches, migraine (with or without aura) and tension headaches;
painful conditions of mixed origin, such as for example chronic back pain including lumbago, or fibromyalgia.

The compounds are also suitable for treating
inflammatory complaints or phenomena caused by sunburn and burns, inflammation of the gums, oedema after burns trauma, cerebral oedema and angiooedema, intestinal complaints including Crohn's disease and ulcerative colitis, irritable bowel syndrome, pancreatitis, nephritis, cystitis (interstitial cystitis), uveitis; inflammatory skin diseases (such as psoriasis and eczema), vascular diseases of the connective tissue, sprains and fractures, and musculoskeletal diseases with inflammatory symptoms such as acute rheumatic fever, polymyalgia rheumatica, reactive arthritis, rheumatoid arthritis, spondylarthritis, and also osteoarthritis, and inflammation of the connective tissue of other origins, and collagenoses of all origins such as systemic lupus erythematodes, scleroderma, polymyositis, dermatomyositis, Sjögren syndrome, Still's disease or Felty syndrome;
inflammatory changes connected with diseases of the airways such as bronchial asthma, including allergic asthma (atopic and non-atopic) as well as bronchospasm on exertion, occupationally induced asthma, viral or bacterial exacerbation of an existing asthma and other non-allergically induced asthmatic diseases;
chronic bronchitis and chronic obstructive pulmonary disease (COPD) including pulmonary emphysema, viral or bacterial exacerbation of chronic bronchitis or chronic obstructive bronchitis, acute adult respiratory distress syndrome (ARDS), bronchitis, lung inflammation, allergic rhinitis (seasonal and all year round), vasomotor rhinitis and diseases caused by dust in the lungs such as aluminosis, anthracosis, asbestosis, chalicosis, siderosis, silicosis, tabacosis and byssinosis, exogenous allergic alveolitis, cystic fibrosis, bronchiectasis, pulmonary diseases in alpha1-antitrypsin deficiency and cough;
diabetes mellitus and its effects (such as e.g. diabetic vasculopathy, diabetic neuropathy, diabetic retinopathy) and diabetic symptoms in insulitis (for example hyperglycaemia, diuresis, proteinuria and increased renal excretion of nitrite and kallikrein);
sepsis and septic shock after bacterial infections or after trauma;
syndromes that cause itching (pruritus) and allergic skin reactions;
damage to the central nervous system;
wounds and tissue damage;
benign prostatic hyperplasia and hyperactive bladder;
vascular diseases such as panarteriitis nodosa, polyarthritis nodosa, periarteriitis nodosa, arteriitis temporalis, Wegner's granulomatosis, giant cell arteriitis, arteriosclerosis and erythema nodosum;
disorders of the motility or spasms of respiratory, genito-urinary, gastro-intestinal including biliary or vascular structures and organs;
post-operative fever;
for the treatment and prevention of cardiovascular diseases such as high blood pressure and related complaints;
for the treatment and prevention of cancer and related complaints;
for the treatment and prevention of psychiatric diseases such as depression;
for the treatment and prevention of urinary incontinence and related complaints;
for the treatment and prevention of morbid obesity and related complaints;
for the treatment and prevention of atherosclerosis and related complaints.

The substances are suitable for causal treatment in the sense of slowing down or stopping the progress of chronically progressive diseases, particularly osteoarthritis, rheumatoid arthritis and spondylarthritis.

In another aspect the present invention encompasses the use of the acid addition salts according to the invention for preparing a medicament for therapeutic use in the above-mentioned indications.

Preferably, the acid addition salts according to the invention are used for the treatment of osteoarthritis, rheumatoid arthritis or COPD.

The term “treatment” or “therapy” refers to a therapeutic treatment of patients with a manifest, acute or chronic indication, including on the one hand symptomatic (palliative) treatment to relieve the symptoms of the disease and on the other hand causal or curative treatment of the indication with the aim of ending the pathological condition, reducing the severity of the pathological condition or delaying the progression of the pathological condition, depending on the nature or gravity of the indication.

The present invention further relates to the use of the acid addition salts according to the invention for preparing a medicament for the acute and prophylactic treatment of acute pain, visceral pain, neuropathic pain, inflammatory/pain receptor-mediated pain, tumour pain, headache pain and pain of mixed causes and other diseases as mentioned above. This use is characterised in that it comprises administering an effective amount of a compound of an acid addition salt according to the invention to a patient requiring such treatment.

The term “patient” preferably refers to a human being.

In addition to their suitability as therapeutic drugs for humans, these substances are also useful in the veterinary medical treatment of domestic pets, exotic animals and farmed animals.

### Combinations

For treating pain, it may be advantageous to combine the compounds according to the invention with stimulating substances such as caffeine or other pain-alleviating active compounds. If active compounds suitable for treating the cause of the pain are available, these can be combined with the compounds according to the invention.

The following compounds may be used for combination therapy, for example:
Non-steroidal antirheumatics (NSAR) such as for example propionic acid derivatives which may be selected from among alminoprofen, bucloxic acid, carprofen, fenoprofen, ibuprofen, ketoprofen, naproxen, oxaprozin, pirprofen, pranoprofen and tiaprofenic acid; acetic acid derivatives which may be selected from among indomethacin, acemetacin, alcofenac, isoxepac, sulindac and tolmetin; fenamic derivatives which may be selected from among meclofenamic acid, mefenamic acid and tolfenamic acid; biphenyl-carboxylic acid derivatives; oxicams which may be selected from among meloxicam, piroxicam and tenoxicam; salicylic acid derivatives which may be selected from among acetylsalicylic and sulphasalazine; pyrazolones which may be selected from among apazone and feprazone); and coxibs which may be selected from among celecoxib and etoricoxib.
Opiate receptor agonists which may for example be selected from among morphine, Darvon, tramadol and buprenorphine;
Cannabinoid agonists such as for example GW-1000;
Sodium channel blockers which may for example be selected from among carbamazepine, mexiletin, pregabalin, tectin and ralfinamide.
N-type calcium channel blockers such as for example ziconotide.
Serotonergic and noradrenergic modulators which may be selected from among for example duloxetine and amitriptyline.
Corticosteroids which may be selected from among for example betamethasone, budesonide, cortisone, dexamethasone, hydrocortisone, methylprednisolone, prednisolone, prednisone and triamcinolone.
Histamine H1-receptor antagonists which may for example be selected from among bromopheniramine, chloropheniramine, dexchloropheniramine, triprolidine, clemastine, diphenhydramine, diphenylpyraline, tripelennamine, hydroxyzine, promethazine, trimeprazine, azatadine, cyproheptadine, antazoline, pheniramine, pyrilamine, loratadine, cetirizine, desloratadine, fexofenadine and levocetirizine.
Leukotriene antagonists and 5-lipoxygenase inhibitors which may for example be selected from among zafirlukast, montelukast, pranlukast and zileuton.
Local anaesthetics which may for example be selected from among ambroxol and lidocaine.
TRVP1 antagonists which may for example be selected from among AZD-1386, JTS-653 and PHE-377.
Nicotine receptor agonists such as for example A-366833.
P2X3-receptor antagonists such as e.g. A-317491.
anti-NGF antibodies and NGF antagonists which may for example be selected from among JNJ-42160443 and PPH 207.
NK1 and NK2 antagonists such as e.g. CP-728663.
NMDA antagonists which may for example be selected from among CNS-5161, AZ-756 and V-3381.
Potassium channel modulators such as e.g. CL-888.
GABA modulators such as e.g. baclofen.
Anti-migraine drugs which may for example be selected from among sumatriptan, zolmitriptan, naratriptan and eletriptan.

The dosage necessary for obtaining a pain-alleviating effect is, in the case of intravenous administration, expediently from 0.01 to 3 mg/kg of body weight, preferably from 0.1 to 1 mg/kg, and, in the case of oral administration, from 0.1 to 8 mg/kg of body weight, preferably from 0.5 to 3 mg/kg, in each case one to three times per day. The compounds prepared according to the invention can be administered intravenously, subcutaneously, intramuscularly, intrarectally, intranasally, by inhalation, transdermally or orally, aerosol formulations being particularly suitable for inhalation. They can be incorporated into customary pharmaceutical preparations, such as tablets, coated tablets, capsules, powders, suspensions, solutions, metered-dose aerosols or suppositories, if appropriate together with one or more customary inert carriers and/or diluents, for example with maize starch, lactose, cane sugar, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances, such as hardened fat, or suitable mixtures thereof.

### EXPERIMENTAL SECTION

### Example 1

### N-(2-hydroxy-ethyl)-4-methoxy-2.6,N-trimethyl-benzenesulphonamide (C)

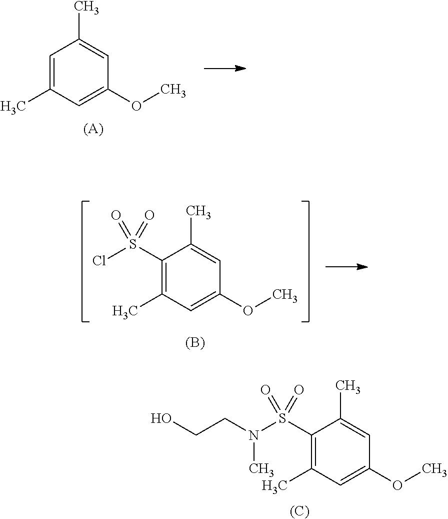

10.00 kg (73.42 mol) of 3,5-dimethylanisol (A) were dissolved in 50.0 L dichloromethane. After cooling to −35° C. a solution of 17.46 kg (149.85 mol) chlorosulphonic acid in 15.0 L dichloromethane was metered in and the mixture was stirred for about another 30 minutes at −35° C.±5° C. After the reaction was complete 40.0 L of water were metered in at −35° C. to 5° C. and then the organic phase was separated off. The organic phase was diluted with 10.0 L dichloromethane, before a solution of 1.23 kg (14.69 mol) of sodium hydrogen carbonate in 29.0 L water was added. After separation of the organic phase and dilution with 10 L of dichloromethane, a solution consisting of 11.58 kg (154.20 mol) of N-methylaminoethanol (B) in 20.0 L dichloromethane was slowly metered in at 10±5° C. After the reaction was complete, a mixture of 42.0 L water and 4.10 kg (42.59 mol) conc. hydrochloric acid was added, starting at 10° C. The organic phase was separated off, diluted with 10.0 L of dichloromethane and the product (C) was totally freed from the solvent in vacuo.

Yield: 14.34 kg (71% of theory)

### Example 2

### {2-[(4-methoxy-2,6-dimethyl-benzenesulphonyl)-methyl-amino]-ethoxy}-acetic acid-sodium salt (E)

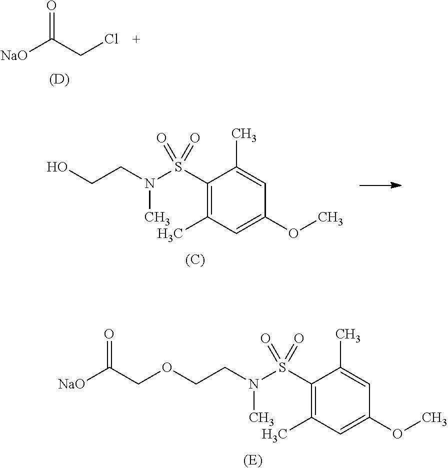

10.00 kg (36.58 mol) of N-(2-hydroxy-ethyl)-4-methoxy-2,6,N-trimethyl-benzenesulphonamide (C) and 6.52 kg (55.97 mol) chloroacetic acid sodium salt (D) were placed in 55.0 L acetonitrile and within about 1 hour 27.99 kg (54.87 mol) of potassium tert. butoxide solution was added at 20±5° C. Then the reaction mixture was stirred for about 1 hour at 20±5° C. After 52.0 L solvent had been distilled off, 60.0 L of toluene were added and a further 52.0 L of solvent were distilled off. Then, 45.0 L water and 6.20 kg (62.92 mol) conc. hydrochloric acid were added and the organic phases were separated off at 50° C. After cooling to 30±5° C., 20.0 L acetone and 2.93 kg (36.58 mol) sodium hydroxide solution (50%) were added and seeded, before the mixture was cooled to 20±5° C. within about 1 hour. After 30 minutes stirring at 20±5° C. the suspension was filtered off, the product (E) was washed twice with acetone and dried.

Yield: 10.91 kg (84% of theory)

### Example 3a

### 2-{2-[(4-methoxy-2,6-dimethyl-benzenesulphonyl)-methyl-amino]-ethoxy}-N-methyl-N-[(1S,3R)-3-(4-methyl-piperazin-1-yl)-cyclohexyl]-acetamide (G′)

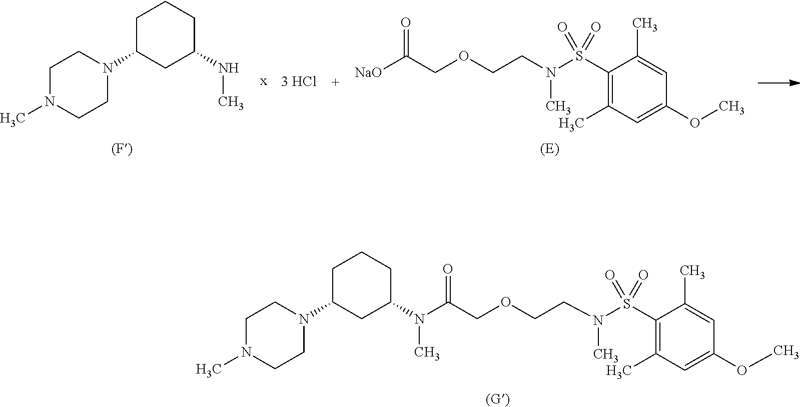

10.00 kg (31.18 mol) methyl-[(1S,3R)-3-(4-methyl-piperazin-1-yl)-cyclohexyl]-amine-tri-hydrochloride (F′) and 12.12 kg (34.30 mol) {2-[(4-methoxy-2,6-dimethyl-benzenesulphonyl)-methyl-amino]-ethoxy}-acetic acid-sodium salt (E) were suspended in 70.0 L toluene and heated to 30° C. Then 61.23 kg (109.13 mol) potassium-tert.-butoxide solution in tetrahydrofuran, 10.0 L toluene, 39.68 kg (62.36 mol) propanephosphonic acid anhydride (50% in tetrahydrofuran) and 10.0 L toluene were metered in successively and the mixture was stirred for about another 30 minutes at 50° C. After the reaction was complete 75.0 L water was added and the pH was adjusted to below 2.0 with concentrated hydrochloric acid. The aqueous phase was separated off, diluted with 15 L of water and at 50° C. it was combined with 80.0 L methylisobutylketone and a mixture of 21.40 kg (267.51 mol) sodium hydroxide solution (50%, industrial grade) and 10.0 L water. The aqueous phase was separated off again before 80.0 L solvent were distilled off in vacuo. The cloudy residue was mixed with 40.0 L ethanol and filtered, before product (G′) was isolated by total elimination of the solvent in vacuo.

Yield: 13.42 kg (82% of theory)

R_{f}=0.45 (CH₂Cl₂/EtOH/NH_{3aq.}=8/2/0.2)

### Example 3b

### 2-{2-[(4-methoxy-2,6-dimethyl-benzenesulphonyl)-methyl-amino]-ethoxy}-N-methyl-N-[(1R,3S)-3-(4-methyl-piperazin-1-yl)-cyclohexyl]-acetamide (G″)

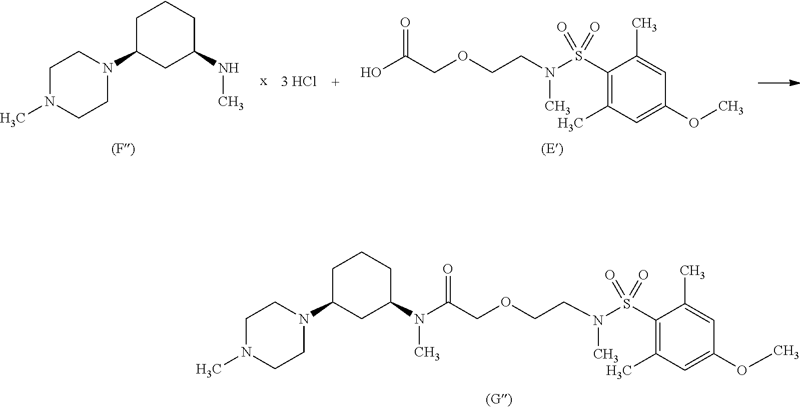

5.00 g (15.6 mmol) methyl-[(1R,3S)-3-(4-methyl-piperazin-1-yl)-cyclohexyl]-amine-tri-hydrochloride (F″) and 5.25 g (15.8 mmol) {2-[(4-methoxy-2,6-dimethyl-benzenesulphonyl)-methyl-amino]-ethoxy}-acetic acid (E′) were suspended in 27.0 mL toluene. Then 35.0 g (78.0 mmol, 25% in toluene) sodium-tert.-amylate solution, followed by 19.9 g (31.25 mmol) propanephosphonic anhydride 50% in ethyl acetate were metered in and the reaction mixture was stirred for about 4 hours at 35±5° C. After the reaction was complete 50 mL water were added. The aqueous phase was separated off and covered with methylisobutylketone. The pH was adjusted to 12.5 by the addition of 4N NaOH, before the organic phase was separated off. After washing with saturated NaCl solution and drying over Na₂SO₄ the solvent was totally eliminated in vacuo and the product (G″) was isolated as a light brown oil.

Yield: 7.5 g (92% of theory)

R_{f}=0.75 (CH₂Cl₂/MeOH/NH_{3aq.}=4/1/0.1)

### Example 4

### Tert-butyl [3-(4-methyl-piperazin-1-yl)-cyclohexyl]-carbamate (J)

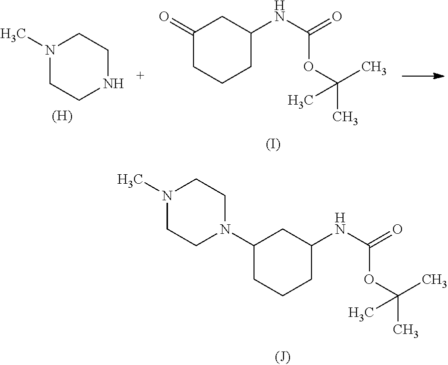

2.6 ml (23.4 mmol) 1-methylpiperazine (H), 1.0 g (4.69 mmol) 3-amino-N-tert-butyloxy-carbonyl-cyclohexanone (I) (AB Chem) and 2.7 ml (49 mmol) glacial acetic acid were dissolved in 10 ml of methanol and stirred for 30 minutes at ambient temperature. Then 1.99 g (9.38 mmol) sodium triacetoxyborohydride were added batchwise and the mixture was stirred for 2 hours at ambient temperature. Then the reaction solution was combined with hydrogen carbonate solution and extracted with dichloromethane. The organic phase was freed from the solvent in vacuo and the residue was chromatographed on RP phase (Varian C18 XRS) (water+5% NH₃/acetonitrile=90:10->0:100).

C₁₆H₃₁N₃O₂ (297.44)

[M+H]+=298

### Example 5

### Methyl-[3-(4-methyl-piperazin-1-yl)-cyclohexyl]-amine (K)

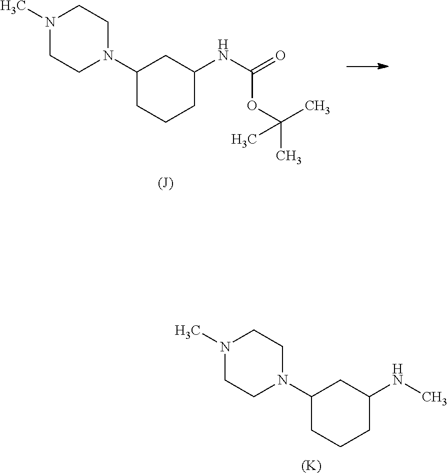

8.57 ml (8.57 mmol) of a 1 M solution of lithium aluminium hydride in toluene were dissolved in 8 ml of THF and at ambient temperature slowly combined with 850 mg (2.86 mmol) of product from Example 4 (J) dissolved in 2 ml of THF. The reaction solution was stirred for 2 hours at 75° C. Then 1N sodium hydroxide solution and water were added. The precipitate was suction filtered and the reaction solution was evaporated to dryness.

C₁₂H₂₅N₃ (211.35)

[M+H]+=212

HPLC: retention time=0.29 min

| | | |
|---|---|---|
| Method: | Column: | Merck Cromolith Speed ROD RP18e, 4.6 × 50 mm |
| | Detection: | 190-400 nm |
| | Eluant A: | water/0.1% formic acid |
| | Eluant B: | acetonitrile/0.1% formic acid |

Gradient:

| time in min | % A | % B | flow rate in mL/min |
|---|---|---|---|
| 0.0 | 90.0 | 10.0 | 1.5 |
| 4.5 | 10.0 | 90.0 | 1.5 |
| 5.0 | 10.0 | 90.0 | 1.5 |
| 5.5 | 90.0 | 10.0 | 1.5 |

### Example 6

### 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-dihydrochloride (1)

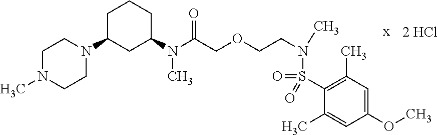

100 mg (0.19 mmol) of 2-{2-[[(4-methoxy-2,6-dimethylphenyl)sulphonyl]-(methyl)amino]-ethoxy}-N-methyl-N-[(1R,3S)-3-(4-methylpiperazin-1-yl)cyclohexyl]acetamide (G′) were dissolved in 1.00 mL ethyl acetate and heated to 50° C. Then 0.13 mL of 2.9M HCl in ethyl acetate (0.38 mmol) were added and the reaction mixture was refluxed. After the addition of 0.10 mL isopropanol the heating bath was removed and the mixture was cooled to ambient temperature. The precipitated product was suction filtered, washed with ethyl acetate and dried.

Yield: 40.0 mg (35% of theory)

### Example 7

### 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2)

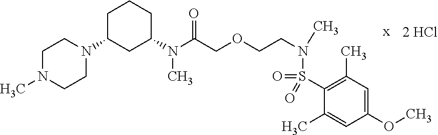

1.00 g (190 mmol) of 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide (G″) were dissolved in 7.00 mL isopropanol and heated to 50° C. Then 1.12 mL 3.4M HCl in isopropanol (381 mmol) were added and the reaction mixture was refluxed. After cooling to ambient temperature the precipitate was filtered off and dried.

Yield: 0.85 g (75% of theory)

### Example 8

### 2-{2-[[(4-methoxy-2,6-dimethylphenyl)sulphonyl]-(methyl)amino]ethoxy}-N-methyl-N-[(1S,3R)-3-(4-methyl piperazin-1-yl)cyclohexyl]acetamide-(L)-tartrate (3)

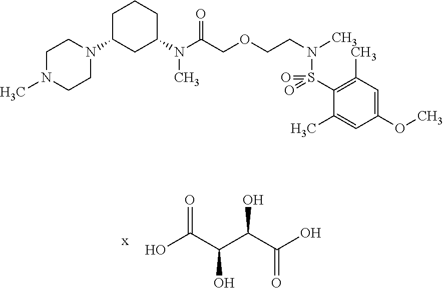

### Variant A:

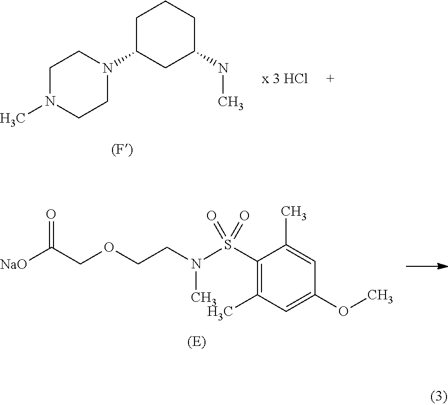

6.00 kg (18.71 mol) of methyl-[(1S,3R)-3-(4-methyl-piperazin-1-yl)-cyclohexyl]amine-tri-hydrochloride (F′) and 7.27 kg (20.58 mol) {2-[(4-methoxy-2,6-dimethyl-benzenesulphonyl)-methyl-amino]-ethoxy}-acetic acid sodium salt (E) were suspended in 33.0 L tetrahydrofuran at 50° C. and 36.74 kg (65.48 mol) of potassium-tert.-butoxide solution in tetrahydrofuran were metered in. Then 23.81 kg (37.41 mol, 50% in tetrahydrofuran) propanephosphonic acid anhydride were added and the reaction mixture was stirred further at 50° C. After the reaction was complete 30.0 L toluene and 33.4 L water were added and the pH of the aqueous phase was adjusted to pH=2.0 with concentrated hydrochloric acid. The aqueous phase was separated off, combined with 48.0 L methylisobutylketone and a mixture of 7.20 kg (89.98 mol, 50%, industrial grade) sodium hydroxide solution and 6.0 L water. After about 5 minutes stirring at 50° C. the aqueous phase was separated off and 48.0 L of solvent were distilled off.

The cloudy residue was combined with 36.0 L of ethanol and filtered. Then the filtrate was heated to 70° C. and a solution of 2.81 kg (18.91 mol) L-(+)-tartaric acid in 24.0 L ethanol was added. After seeding and the onset of crystallisation the suspension was cooled further for about 1 hour at 70° C. and cooled to 20° C. Then the product (3) was separated off, washed and dried.

Yield: 11.3 kg (90% of theory)

melting point: 167° C.±5° C.

### Variant B:

64.0 mg (0.12 mmol) of 2-{2-[[(4-methoxy-2,6-dimethylphenyl)sulphonyl]-(methyl)amino]-ethoxy}-N-methyl-N [(1S,3R)-3-(4-methylpiperazin-1-yl)cyclohexyl]acetamide (G″) were dissolved in 0.11 mL EtOH and 0.38 mL isobutyl-methyl-ketone and heated to 50° C. Then 18.3 mg (0.12 mmol) of L-(+)-tartaric acid dissolved in 0.27 ml of ethanol were added and the mixture was heated to 65-82° C. After slow cooling to ambient temperature the product was filtered off, washed with isobutylmethylketone and dried.

Yield: 78.2 mg (95% of theory)

melting point: 167° C.±5° C.

enantiomeric purity: 99.6%

### Example 9

### 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4)

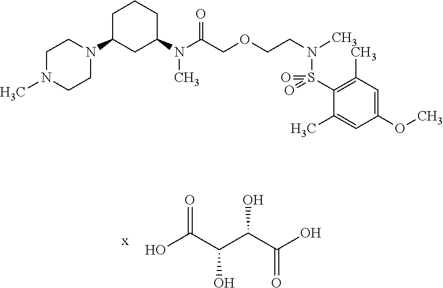

100 mg (0.19 mmol) of 2-{2-[[(4-methoxy-2,6-dimethylphenyl)sulphonyl]-(methyl)amino]-ethoxy}-N-methyl-N-[(1R,3S)-3-(4-methylpiperazin-1-yl)cyclohexyl]acetamide (G′) were dissolved in 1.00 mL acetone and heated to 50° C. Then 28.6 mg (0.19 mmol) of D-(−)-tartaric acid and 1.00 ml of ethanol were added and the mixture was heated. After slowly cooling to ambient temperature the product was filtered off, washed with acetone and dried.

Yield: 70.0 mg (54% of theory)

melting point: 167±5° C.

### Example 10

### 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-difumarate (5)

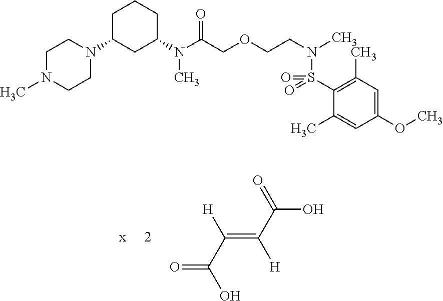

5.00 g (9.53 mmol) of 2-{2-[[(4-methoxy-2,6-dimethylphenyl)sulphonyl]-(methyl)amino]-ethoxy}-N-methyl-N[(1S,3R)-3-(4-methylpiperazin-1-yl)cyclohexyl]acetamide were dissolved in 10 mL methanol and heated to 50° C. Then the solution of 2.21 g (19.06 mmol) fumaric acid in 10 mL of hot methanol was added and the reaction mixture was cooled to ambient temperature. The precipitate was filtered off, dried and suspended again in hot acetonitrile. After filtration and drying the product was obtained.

Yield: 2.16 g (30% of theory)

melting point: 153±5° C.

### Example 11

### 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-difumarate (6)

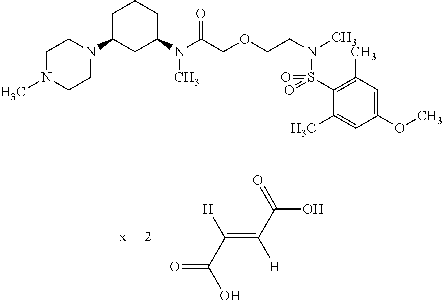

165 mg (0.32 mmol) of 2-{2-[[(4-methoxy-2,6-dimethylphenyl)sulphonyl]-(methyl)amino]-ethoxy}-N-methyl-N [(1R,3S)-3-(4-methylpiperazin-1-yl)cyclohexyl]acetamide were dissolved in 0.10 mL methanol and heated to 50° C. Then 73.0 mg (0.64 mmol) fumaric acid suspended in 0.75 ml of methanol were added and the mixture was heated to 50-64° C. After cooling to ambient temperature the solvent was removed and the residue was dissolved in 0.4 mL of warm 1-butanol. After cooling to ambient temperature the product was filtered off, washed with 1-butanol and dried.

Yield: 175 mg (74% of theory)

melting point: 152±5° C.

### BRIEF DESCRIPTION OF THE FIGURES

FIG. 1 shows the X-ray powder diffractogram of the crystalline compound 2-{2-[[(4-methoxy-2,6-dimethylphenyl)sulphonyl]-(methyl)amino]ethoxy}-N-methyl-[(1R,3S)-3-(4-methylpiperazin-1-yl)cyclohexyl]acetamide-dihydrochloride (1).

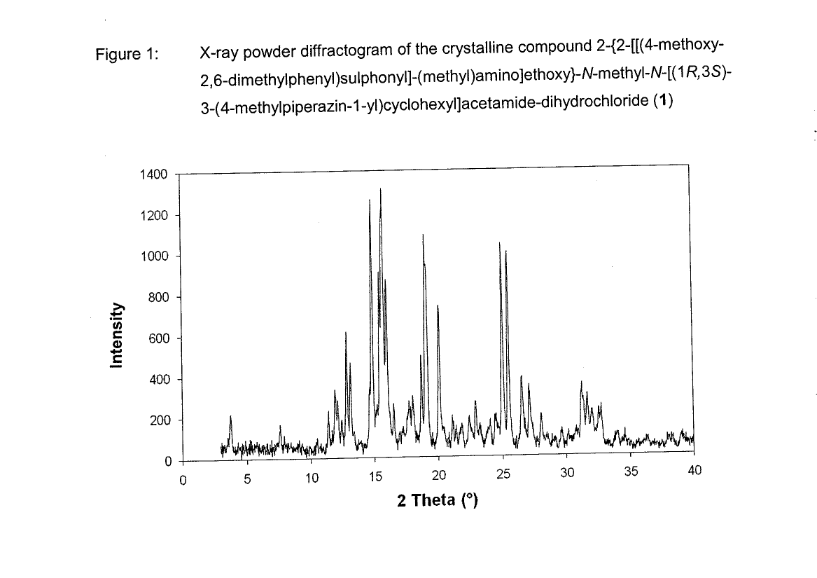

FIG. 2 shows the X-ray powder diffractogram of the crystalline compound 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2).

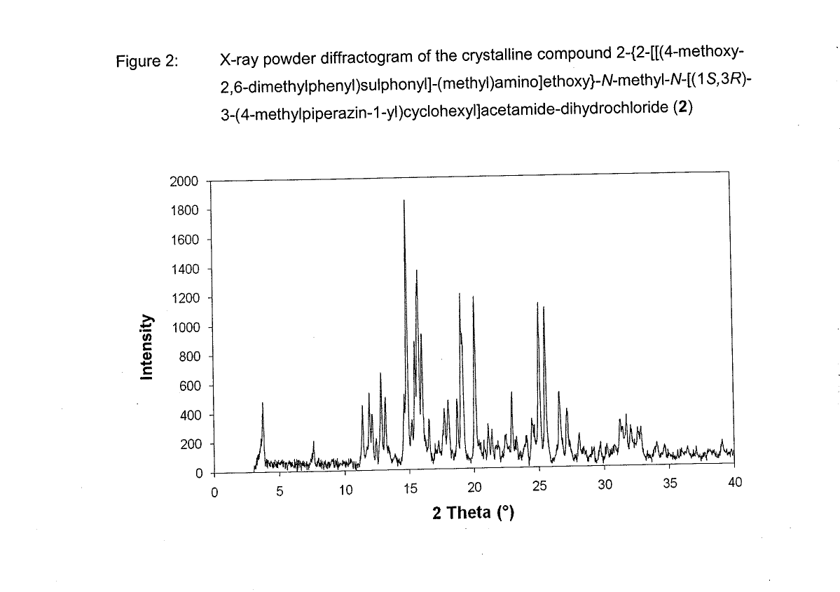

FIG. 3 shows the X-ray powder diffractogram of the crystalline compound 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3).

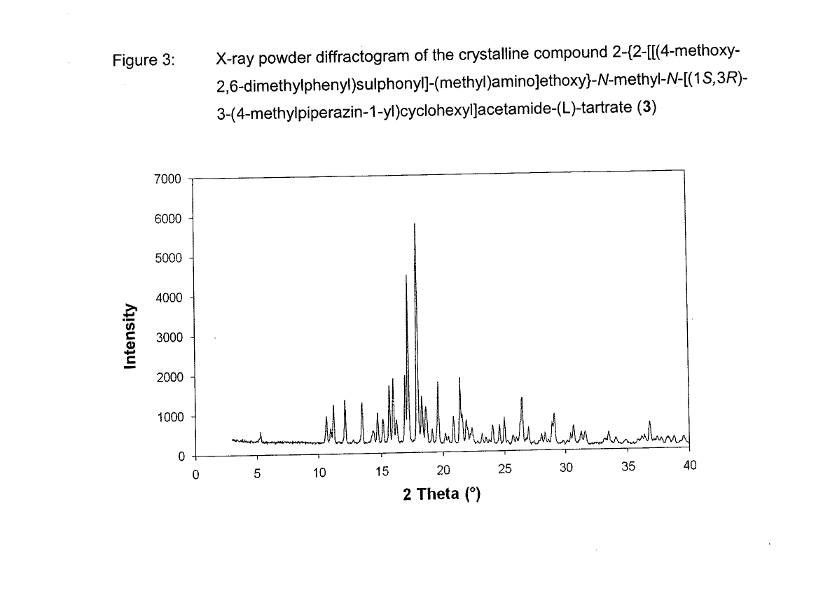

FIG. 4 shows the X-ray powder diffractogram of the crystalline compound 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4).

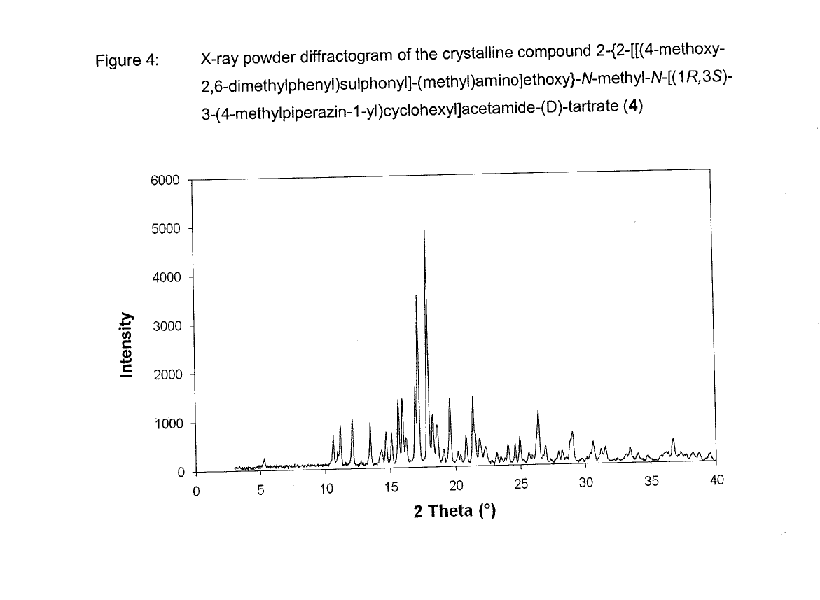

1. An acid addition salt AB of the following free base of formula A 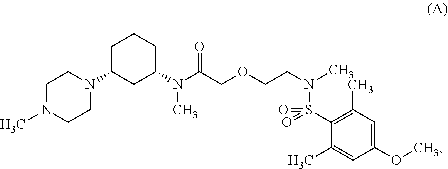 [SMILES: COc1cc(C)c(S(=O)(=O)N(C)CCOCC(=O)N(C)[C@H]2CCC[C@@H](N3CCN(C)CC3)C2)c(C)c1]
or the enantiomer thereof with a physiologically acceptable acid B, which is selected from the group consisting of hydrochloric acid, fumaric acid and tartaric acid.

2. An acid addition salt according to claim 1, in crystalline form.

3. A crystalline acid addition salt AB according to claim 1, selected from the group consisting of:
(1) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-dihydrochloride (1),
(2) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2),
(3) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3),
(4) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4),
(5) 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-difumarate (5) and
(6) 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-difumarate (6).

4. The crystalline compound (3) according to claim 3, characterised by a melting point of T_{mp.}=167±5° C.

5. The crystalline compound (4) according to claim 3, characterised by a melting point of T_{mp.}=167±5° C.

6. The crystalline compound (5) according to claim 3, characterised by a melting point of T_{mp.}=153±5° C.

7. The crystalline compound (6) according to claim 3, characterised by a melting point of T_{mp.}=152±5° C.

8. Crystalline 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-dihydrochloride (1), characterised in that in the X-ray powder diagram it has inter alia the characteristic values d=5.95 Å, 5.62 Å and 4.65 Å.

9. Crystalline 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-dihydrochloride (2), characterised in that in the X-ray powder diagram it has inter alia the characteristic values d=5.95 Å, 5.62 Å and 4.65 Å.

10. Crystalline 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3), characterised in that in the X-ray powder diagram it has inter alia the characteristic values d=4.94 Å, 5.15 Å and 5.22 Å.

11. Crystalline 2-{2-[[(4-methoxy-2,6-dimethylphenyl)sulphonyl]-(methyl)amino]ethoxy}-N-methyl-N-[(1R,3S)-3-(4-methylpiperazin-1-yl)cyclohexyl]acetamide-(D)-tartrate (4), characterised in that in the X-ray powder diagram it has inter alia the characteristic values d=4.94 Å, 5.15 Å and 5.22 Å.

12. Crystalline 2-{2-[[(4-methoxy-2,6-dimethylphenyl)sulphonyl]-(methyl)amino]ethoxy}-N-methyl-N-[(1S,3R)-3-(4-methylpiperazin-1-yl)cyclohexyl]acetamide-(L)-tartrate (3), characterised in that the crystals are present in an orthorhombic system.

13. A pharmaceutical composition comprising a compound according to one of claims 1 to 12 together with one or more inert carriers and/or diluents.

14-16. (canceled)

17. A process for preparing an acid addition salt AB according to one of claims 1 to 12, comprising the steps of:
(a) reacting 3,5-dimethylanisol of formula II 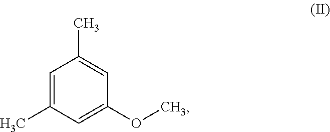
with chlorosulphonic acid;
(b) reacting 4-methoxy-2,6-dimethylsulphonyl chloride of formula III obtained in step (a) 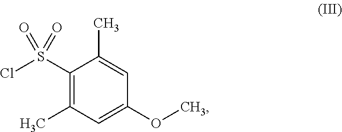
with the compound of formula IV 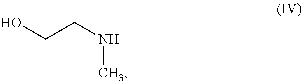
(c) reacting the compound of formula V obtained in step (b) 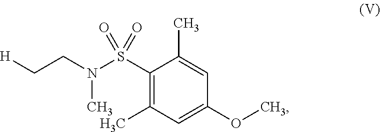 [SMILES: CCN(C)S(=O)(=O)c1c(C)cc(OC)cc1C]
with a compound of general formula VI 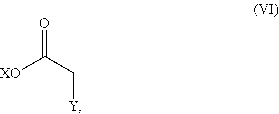
wherein X denotes a hydrogen atom, an alkali metal, for example lithium, sodium or potassium, or a C₁₋₄-alkyl group, but preferably sodium, and Y denotes a halogen atom, for example chlorine or bromine, preferably chlorine;
(d) optionally recrystallising a compound of general formula VII obtained in step (c) 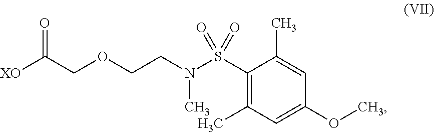
wherein X denotes a hydrogen atom, an alkali metal, for example lithium, sodium or potassium, or a C₁₋₄-alkyl group, but preferably sodium, from a solvent;
(e) coupling a compound of general formula VIII obtained in step (c) or (d) 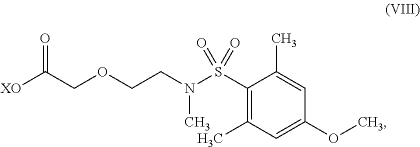
wherein X denotes a hydrogen atom, an alkali metal, for example lithium, sodium or potassium, or a C₁₋₄-alkyl group, but preferably sodium, with a compound of formula IX 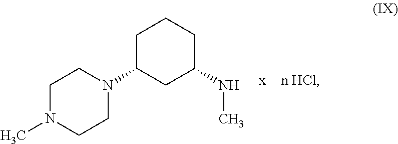
or the enantiomer thereof, wherein n denotes one of the numbers 0, 1, 2 or 3;
(f) optionally isolating a compound of formula A obtained in step (d) 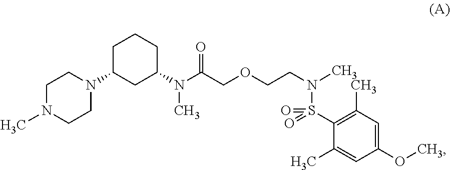 [SMILES: COc1cc(C)c(S(=O)(=O)N(C)CCOCC(=O)N(C)[C@H]2CCC[C@@H](N3CCN(C)CC3)C2)c(C)c1]
or the enantiomer thereof;
(g) dissolving the compound of formula A obtained in step (e) or (0, or the enantiomer thereof, in a polar solvent;
(h) adding a physiologically acceptable acid B selected from among hydrochloric acid, fumaric acid and tartaric acid, optionally dissolved in a polar solvent;
(i) crystallising out an acid addition salt AB, optionally by the addition of an antisolvent, by cooling, distillation or seeding.
